(19) United States
(12) Patent Application Publication
(10) Pub. No.: US 2026/0224390 A1
(43) Pub. Date: Aug. 6, 2026

(54) PORTABLE NECK PILLOW WITH AN ANTI-SNORING ASSEMBLY

(71) Applicant: Zhejiang Youdian Medical Technology Co., Ltd., Ningbo (CN)

(72) Inventors: Yueping ZHU, Ningbo (CN); Xin GUO, Ningbo (CN)

(73) Assignee: Zhejiang Youdian Medical Technology Co., Ltd., Ningbo (CN)

(21) Appl. No.: 19/312,032

(22) Filed: Aug. 27, 2025

(30) Foreign Application Priority Data
Jan. 20, 2025 (CN) 2025100878620

(51) Int. Cl. A61F 5/56; A47G 9/00; A47G 9/10; A61H 23/02

(52) U.S. Cl. CPC A61F 5/56; A47G 9/1027; A47G 9/1045; A47G 9/1081; A61H 23/0263; A47G 2009/003; A61H 2201/0103; A61H 2201/1611; A61H 2201/5048; A61H 2201/5056; A61H 2201/5071; A61H 2201/5092; A61H 2230/405

(57) ABSTRACT

Disclosed is a portable neck pillow with an anti-snoring assembly, relating to the technical field of nursing equipment. The portable neck pillow includes a pillow main body and a control module. The anti-snoring assembly includes a vibration unit and an inflation and deflation unit. The inflation and deflation unit includes an airbag element, an air pump, and an air path part connecting the airbag element and the air pump. And, the anti-snoring assembly also includes a first judgment unit and a second judgment unit, the first judgment unit being for judging a snoring state of a user, and the second judgment unit being for judging a head deflection direction of the user. The control module includes a power supply and a control system. The control system controls movement of the vibration unit and the inflation and deflation unit according to the first judgment unit and the second judgment unit.

### CROSS-REFERENCE TO RELATED APPLICATIONS

The application claims priority to Chinese patent application No. 2025100878620, filed on Jan. 20, 2025, the entire contents of which are incorporated herein by reference.

### TECHNICAL FIELD

The present disclosure relates to the technical field of nursing equipment, and in particular, relates to a portable neck pillow with an anti-snoring assembly.

### BACKGROUND

The global neck pillow market size was 1.42 billion USD in 2023, and is expected to reach 2.25 billion USD by 2032, with a compound annual growth rate of 5.3% during a forecast period. The neck pillow market is growing at a significant rate, mainly driven by the following several key factors. An increasing awareness among people of benefits of neck pillows for sleep quality and relieving pain, coupled with an increasing popularity of travel and outdoor leisure activities, leads to a rising demand for portable, lightweight neck cushions. With a surge in remote work, and a prevalence of neck pain and musculoskeletal diseases, people are seeking solutions to improve comfort.

Snoring is a problem faced by many people, not only affecting an own sleep quality of a person, but also disturbing a rest of others. Snoring, especially in a business trip environment and a travel environment, extremely affects a work state and a mood of other people. Therefore, developing a portable neck pillow that is capable of both providing comfortable neck support and effectively stopping snoring has significant practical importance.

Patent CN201820374638.5 discloses a neck pillow, the neck pillow comprising: a neck pillow body, a controller, a massage module, and a physiological signal detection module; the physiological signal detection module, the massage module and the controller are disposed in the neck pillow body; the massage module and the physiological signal detection module are connected to the controller; the physiological signal detection module collects physiological signal data and transmits the physiological signal data to the controller, and the controller controls the massage module to work according to the physiological signal data. Through an additionally provided physiological signal detection module, real-time detection of a state of a user during use by the neck pillow can be realized, enabling the neck pillow to make changes in massage intensity, massage method, and the like following changes in physiological signals. A defect of the solution is that: simply reminding the user through the massage module easily affects a sleep state, causing the user to wake up multiple times, without actively adjusting a head posture of the user or providing a support force.

Patent CN202210398726.X discloses an improved inflatable neck pillow, comprising a housing, and a main unit disposed in the housing. Two ends of the housing are each connected with an extension airbag. When the extension airbags are inflated and expanded, the housing and the two extension airbags form a U-shape. The main unit comprises a rear base, a pivot shaft, a flip base movably sleeved on the pivot shaft, and a massage assembly disposed on the flip base. An ejection airbag is disposed between the rear base and the flip base. The main unit further comprises a plurality of inflation devices respectively for inflating the extension airbags and the ejection airbag. Defects of the solution are that: first, an adjustment of airbag inflation and deflation is used for wrapping a neck, does not have a function of adjusting a head direction, and excessively fitting the neck will cause user discomfort; and second, structurally complex and heavy components such as a reverse base are provided, increasing a weight of the neck pillow, making the neck pillow not only inconvenient for long-term wearing but also inconvenient to be picked up and carried.

In summary, existing neck pillows have the following problems: first, a structure of a provided massage structure or a vibration structure is complex and has a large weight, causing pressure on a neck; second, it is difficult to provide a support force for a user whose head tilts while asleep, that is, a neck-cheek junction area cannot be supported; and third, it is difficult to balance both light weight and multifunctionality.

### SUMMARY

An object of the present disclosure is to provide a portable neck pillow with an anti-snoring assembly, capable of monitoring a snoring state and a head deflection direction of a user in real time, and effectively alleviating snoring symptoms through a synergistic effect of a vibration unit and an inflation and deflation unit, while having portability and comfort.

To solve the above technical problems, the present application is solved by the following technical solution: a portable neck pillow with an anti-snoring assembly, comprising a pillow main body and a control module, where the anti-snoring assembly comprises a vibration unit and an inflation and deflation unit, the inflation and deflation unit comprises an airbag element, an air pump, and an air path part connecting the airbag element and the air pump; and, the anti-snoring assembly further comprises a first judgment unit and a second judgment unit, the first judgment unit is used for judging a snoring state of a user, the second judgment unit is used for judging a head deflection direction of the user, the control module comprises a power supply and a control system, and the control system controls movement of the vibration unit and the inflation and deflation unit according to the first judgment unit and the second judgment unit.

In an embodiment, the vibration unit and the inflation and deflation unit move in coordination with each other.

In an embodiment, the airbag element is in a split-type connection with the pillow main body, the pillow main body is sleeved with a first kit, the airbag element is at least partially sleeved with a second kit, and at least a partial connection area between the first kit and the second kit is a detachable connection; and, the airbag element is spatially located above the pillow main body.

In an embodiment, the second kit covers above the airbag element, and when a head of a user deflects to a left side or a right side, the second kit abuts against a neck-cheek junction area of the user. At this time, inflating the first airbag or the second airbag that is subjected to pressure can support the neck-cheek junction area.

In an embodiment, the second kit is capable of being detached for cleaning.

In an embodiment, the pillow main body comprises an inner ring part and an outer ring part, a left side and a right side of the outer ring part are respectively provided with a first control compartment and a second control compartment, the air pump is fixed in the first control compartment, the power supply is fixed in the second control compartment, and, the control module further comprises a button, the button is connected to the second control compartment, the button is used for switching a neck pillow mode, and the neck pillow comprises at least a vibration function and an inflation function.

In an embodiment, the first control compartment further comprises an electric control valve.

In an embodiment, the electric control valve is a three-way valve.

In an embodiment, the vibration unit comprises a plurality of eccentric vibration motors and an attachment box for fixing the eccentric vibration motors, the attachment box comprises at least one arc-shaped surface, and the arc-shaped surface abuts against a rear neck position of the user to ensure effective transmission of a vibration effect.

In an embodiment, the pillow main body is a pillow core and is made of a memory foam material.

In an embodiment, the vibration unit comprises two eccentric vibration motors.

In an embodiment, the airbag element comprises a first airbag and a second airbag, and the first airbag and the second airbag are connected through a connection segment; and, the air path part comprises an air tube, an air nozzle is disposed below the first airbag and the second airbag, and the air nozzle is connected to the air tube.

In an embodiment, the first airbag and the second airbag are arc-shaped as a whole.

In an embodiment, the first kit comprises a through hole, the air tube passes through the through hole and is then connected to the air pump along the outer ring part; and, the inflation and deflation unit further comprises an electric control valve, capable of precisely controlling a flow of gas.

In an embodiment, the first judgment unit comprises a microphone sensor and a data acquisition collector, and the control system controls the first judgment unit to perform real-time voiceprint recognition; and, when the first judgment unit determines that the user is in the snoring state, the control system drives the vibration unit to perform vibration and/or drives the air pump to cause the airbag element to perform inflation and deflation, so as to change a sleep posture of the user and alleviate snoring.

In an embodiment, the second judgment unit comprises a pressure sensor, and the pressure sensor is disposed on the inner ring part; and, when the head of the user deflects toward the left or toward the right, the pressure sensor is subjected to pressure, and the control system drives the air pump to cause the airbag element to perform inflation.

In an embodiment, the second judgment unit comprises an infrared sensor, and the infrared sensor is disposed on the outer ring part; and, when the head of the user deflects toward the left or toward the right, the pillow main body deforms, a detection area of the infrared sensor deforms, and the control system drives the air pump to cause the airbag element to perform inflation.

In an embodiment, the portable neck pillow further comprises a balance box, the balance box is disposed in a middle area of the outer ring part, and both the balance box and the attachment box are located on a center line of the pillow body.

In an embodiment, the portable neck pillow further comprises a heating unit, the heating unit is disposed on the inner ring part, and the control system drives a working state of the heating unit.

In an embodiment, a pathway exists between the balance box and the attachment box for placing electric wires.

Compared with a prior art, advantages of the present disclosure are as follows:
1. In the prior art, a neck pillow cannot both stop snoring and support a neck-cheek junction area of a user. The neck pillow of the present disclosure, by virtue of a collaborative operation of the first judgment unit and the second judgment unit, achieves accurate and real-time monitoring of a snoring state and a head posture of the user. Once the first judgment unit determines that the user is in the snoring state through the microphone sensor and the data acquisition collector, the control system rapidly reacts, driving the vibration unit and/or the inflation and deflation unit to work. The eccentric vibration motors of the vibration unit, through the attachment box having the arc-shaped surface that abuts against the rear neck of the user, accurately transmit mild vibration to the rear neck of the user, cleverly reminding the user to naturally adjust a neck posture, that is, waking up the user after vibration, thereby effectively alleviating snoring symptoms. At the same time, the inflation and deflation unit, according to a judgment result of the second judgment unit on the head deflection direction of the user, timely performs an inflation and deflation operation on the airbag element. For example, when the pressure sensor or the infrared sensor in the second judgment unit detects that the head of the user deflects toward the left, the air pump timely inflates the first airbag, cleverly adjusting a support force and a support angle of the neck pillow for the neck of the user, trying to turn the head of the user from a tilted state to an upright state, so that the head returns to a position more conducive to keeping an airway unobstructed, thereby fundamentally reducing an occurrence probability of snoring, and thus improving a sleep quality or a rest state of the user and people around the user. On the other hand, even if the user does not snore, the inflation and deflation unit can also provide a support force to a lower jaw and a cheek of the user under the control of the second judgment unit, making the user feel more comfortable and preventing snoring to a certain extent.
2. Different from the prior art, the airbag element and the pillow main body adopt a split-type connection, and the connection area of the first kit and the second kit is detachable. This design greatly facilitates daily cleaning and maintenance of the neck pillow. A user only needs to perform a simple operation to separate the airbag element from the pillow main body and perform comprehensive cleaning on the airbag element and the pillow main body respectively. In particular, the second kit, as a part that directly contacts the neck of the user, can be separately detached for deep cleaning, effectively removing dirt and bacteria, ensuring that the neck pillow is always kept clean and hygienic, and providing the user with a healthy and comfortable usage experience. Moreover, this split-type design makes replacement of various components more convenient. When a certain component is damaged, the user does not need to replace the entire neck pillow, but only needs to replace a corresponding damaged component, which greatly reduces a usage cost and prolongs a product life of the product.
3. Different from the prior art, a control module layout of the neck pillow of the present disclosure is rational, and the button is disposed on the second control compartment, which is convenient for the user to operate at any time. The user can easily switch a working mode of the neck pillow through the button, such as turning on or off the vibration function, adjusting an inflation amount to meet different support needs, etc. And, the three-way valve in the first control compartment cooperates closely with the air path part to accurately control a gas flow speed and a gas flow rate, achieving precise regulation of the inflation and deflation process of the airbag element. This precise control not only improves an operation experience of the user with the neck pillow, but also flexibly adjusts a support state of the neck pillow according to actual needs of the user, adapting to users with different needs, and satisfying users with different neck proportions.
4. Different from the prior art, the balance box and the attachment box of the present disclosure are both located on the center line of the pillow body. The balance box is disposed in the middle area of the outer ring part, effectively enhancing an overall stability of the neck pillow during use. No matter how the user changes posture during sleep or rest, the neck pillow can always maintain balance and provide the user with stable support. On the other hand, the arc-shaped surface of the attachment box perfectly abuts against the rear neck of the user, which not only ensures effective transmission of the vibration effect, but also provides comfortable support when the user wears the neck pillow, reducing neck pressure, different from a roller-type massage device. In addition, the first airbag and the second airbag of the airbag element are designed to be arc-shaped as a whole, and when the head of the user deflects to the left side or the right side, the second kit is capable of abutting against the neck-cheek junction area of the user, and supporting the area by inflating a corresponding airbag, further improving comfort of the neck pillow, and creating a more pleasant sleep environment for the user. Finally, the second kit can also be detached, which is convenient for the user to clean. The user can also freely choose whether to use the airbags, or choose airbags of what height, providing strong selectivity.

In summary, the portable neck pillow with the anti-snoring assembly of the present disclosure, through innovative designs in multiple aspects, exhibits significant advantages in improving sleep quality, facilitating maintenance, optimizing operation experience, enhancing stability and comfort, and innovative functions, providing users with a powerful, comfortable, and convenient portable sleep auxiliary equipment.

### BRIEF DESCRIPTION OF DRAWINGS

FIG. 1 is a schematic diagram of an overall structure of a portable neck pillow with an anti-snoring assembly of the present disclosure, showing a position relationship of a pillow main body, an airbag element, and a balance box, etc., where the pillow main body comprises an inner ring part and an outer ring part, the airbag element is located above the pillow main body, and is connected through a first kit and a second kit.

FIG. 2 is a schematic diagram of a connection structure of the pillow main body and the airbag element, showing the first kit sleeved on the pillow main body and a second kit at least partially sleeved on the airbag element.

FIG. 3 is a schematic diagram of a first control compartment and a second control compartment on a left side and a right side of the outer ring part.

FIG. 4 is a schematic diagram of a connection structure of the airbag element and an air path part, showing that the airbag element comprises a first airbag and a second airbag, the first airbag and the second airbag are connected through a connection segment, and an air nozzle is disposed below the first airbag and the second airbag, and the air nozzle is connected to an air tube.

FIG. 5 is a schematic diagram of an inflated state of the airbag element above the pillow main body.

FIG. 6 is a layout schematic of the balance box and an attachment box, where a pathway exists between the two.

FIG. 7 is a principle diagram of the neck pillow supporting a neck of a user to return a head to a position more conducive to keeping an airway unobstructed.

FIG. 8 shows another embodiment of the present application, where the airbag element is below the pillow main body.

The names of the parts indicated by the respective numbers in the drawings are as follows:
1—pillow main body (1), 11—inner ring part (11), 12—outer ring part (12), 2—control module (2), 21—power supply (21), 22—control system (22), 23—button (23), 3—anti-snoring assembly (3), 31—vibration unit (31), 311—eccentric vibration motor (311), 312—attachment box (312), 3121—arc-shaped surface (3121), 32—inflation and deflation unit (32), 321—airbag element (321), 3211—first airbag (3211), 3212—second airbag (3212), 3213—connection segment (3213), 322—air pump (322), 323—air path part (323), 324—electric control valve (324), 33—first judgment unit (33), 34—second judgment unit (34), 4—first kit (4), 5—second kit (5), 6—first control compartment (6), 7—second control compartment (7), 8—balance box (8).

### DETAILED DESCRIPTION OF THE EMBODIMENTS

In the following description of the drawings and specific embodiments, details of one or more embodiments of the present application will be set forth. Other features, objects, and advantages of the present application will be apparent from the description, the drawings, and the claims.

It should be understood that the phrases and terms used herein are for the purpose of description and should not be regarded as limiting. The use of “including,” “comprising,” or “having” and variations thereof herein is intended to be open-ended, encompassing the items listed thereafter and equivalents thereof as well as additional items.

The present application will be described in more detail below with reference to different embodiments and examples of several aspects of the present application.

In the present application, the term “inner” or “inner ring” refers to a side or a circle that abuts against a neck, and the term “outer” or “outer ring” refers to a side or a circle that does not abut against the neck.

In the present application, the term “up” or “upward” refers to an upward direction when a user wears the neck pillow, and the term “down” or “downward” refers to a downward direction when the user wears the neck pillow.

### Specific Embodiments

### Embodiment One

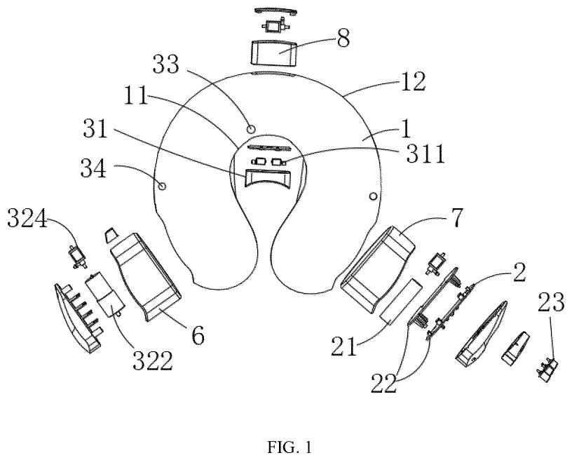

This embodiment provides a portable neck pillow with an anti-snoring assembly (3), as shown in FIG. 1, comprising a pillow main body (1) and a control module (2). The anti-snoring assembly (3) comprises a vibration unit (31) and an inflation and deflation unit (32). The inflation and deflation unit (32) comprises an airbag element (321), an air pump (322), and an air path part (323) connecting the airbag element (321) and the air pump (322). And, the anti-snoring assembly (3) further comprises a first judgment unit (33) and a second judgment unit (34). The first judgment unit (33) is used for judging a snoring state of a user. The second judgment unit (34) is used for judging a head deflection direction of the user. The control module (2) comprises a power supply (21) and a control system (22). The control system (22) controls movement of the vibration unit (31) and the inflation and deflation unit (32) according to the first judgment unit (33) and the second judgment unit (34).

In Embodiment One, the vibration unit (31) and the inflation and deflation unit (32) cooperate with each other in movement.

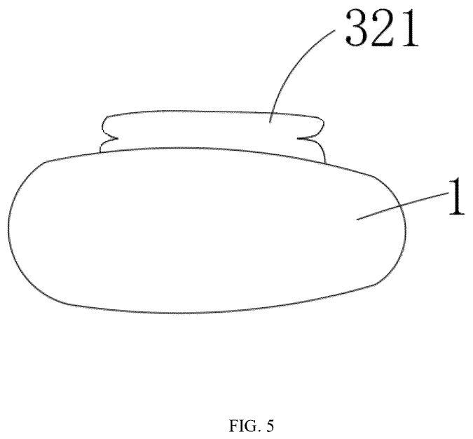

In Embodiment One, the airbag element (321) and the pillow main body (1) are in a split-type connection. The pillow main body (1) is sleeved with a first kit (4). The airbag element (321) is at least partially sleeved with a second kit (5). At least a partial connection area between the first kit (4) and the second kit (5) is a detachable connection. And, the airbag element (321) is spatially located above the pillow main body (1), as shown in FIG. 5.

In Embodiment One, the second kit (5) covers above the airbag element (321). When a head of the user deflects to a left side or a right side, the second kit (5) abuts against a neck-cheek junction area of the user. At this time, inflating the first airbag (3211) or the second airbag (3212) that is subjected to pressure is capable of supporting the neck-cheek junction area.

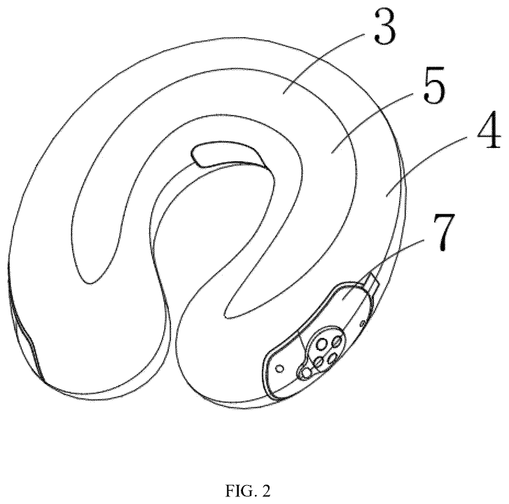

In Embodiment One, the second kit (5) is capable of being detached for cleaning, as shown in FIG. 2.

In Embodiment One, the pillow main body (1) comprises an inner ring part (11) and an outer ring part (12). A left side and a right side of the outer ring part (12) are respectively provided with a first control compartment (6) and a second control compartment (7). The air pump (322) is fixed in the first control compartment (6). The power supply (21) is fixed in the second control compartment (7). And, the control module (2) further comprises a button (23). The button (23) is connected to the second control compartment (7). The button (23) is used for switching a neck pillow mode. The neck pillow comprises at least a vibration mode and an inflation mode. The vibration mode is used for massaging a neck. The inflation mode is used for supporting the head and the neck of the user.

In Embodiment One, the first control compartment (6) further comprises an electric control valve (324).

In Embodiment One, the vibration unit (31) comprises a plurality of eccentric vibration motors (311) and an attachment box (312) for fixing the eccentric vibration motors (311). The attachment box (312) comprises at least one arc-shaped surface (3121). The arc-shaped surface (3121) abuts against a rear neck position of the user to ensure effective transmission of a vibration effect.

In Embodiment One, the vibration unit (31) comprises two eccentric vibration motors (311).

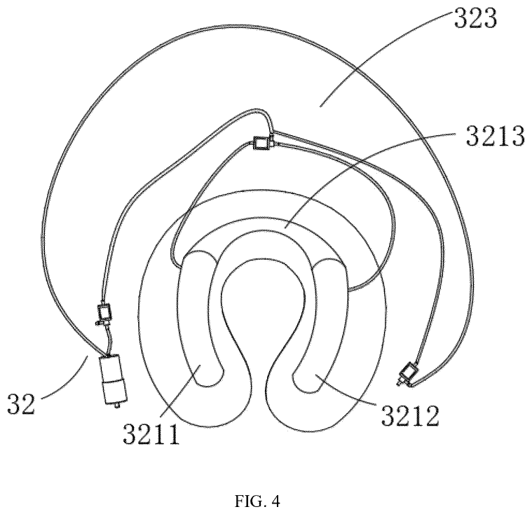

In Embodiment One, the airbag element (321) comprises a first airbag (3211) and a second airbag (3212). The first airbag (3211) and the second airbag (3212) are connected through a connection segment (3213). And, the air path part (323) comprises an air tube. An air nozzle is disposed below the first airbag (3211) and the second airbag (3212). The air nozzle is connected to the air tube, as shown in FIG. 4.

In Embodiment One, the first airbag (3211) and the second airbag (3212) are a double-layer airbag.

In Embodiment One, the first airbag (3211) and the second airbag (3212) are arc-shaped as a whole.

In Embodiment One, the first kit (4) comprises a through hole. The air tube passes through the through hole and is then connected to the air pump (322) along the outer ring part (12). And, the inflation and deflation unit (32) further comprises the electric control valve (324), which is capable of precisely controlling a flow of gas.

In Embodiment One, the first judgment unit (33) comprises a microphone sensor and a data acquisition collector. The control system (22) controls the first judgment unit (33) to perform real-time voiceprint recognition. And, when the first judgment unit (33) determines that the user is in the snoring state, the control system (22) drives the vibration unit (31) to perform vibration and/or drives the air pump (322) to cause the airbag element (321) to perform inflation and deflation, so as to change a sleep posture of the user and alleviate snoring.

In Embodiment One, the second judgment unit (34) comprises a pressure sensor. The pressure sensor is disposed on the inner ring part (11). And, when the head of the user deflects toward the left or toward the right, the pressure sensor is subjected to pressure, and the control system (22) drives the air pump (322) to cause the airbag element (321) to perform inflation.

In Embodiment One, the second judgment unit (34) comprises an infrared sensor. The infrared sensor is disposed on the outer ring part (12). And, when the head of the user deflects toward the left or toward the right, the pillow main body (1) deforms, a detection area of the infrared sensor deforms, and the control system (22) drives the air pump (322) to cause the airbag element (321) to perform inflation.

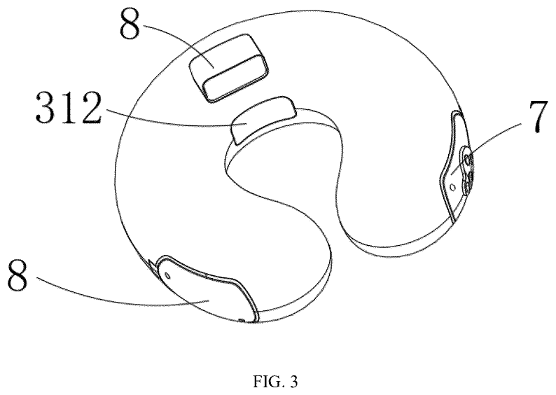

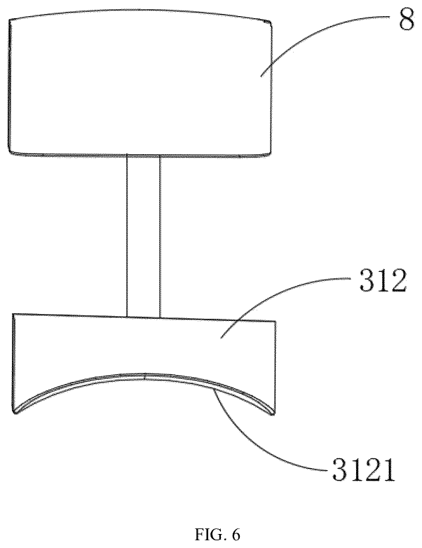

In Embodiment One, the portable neck pillow further comprises a balance box. The balance box (8) is disposed in a middle area of the outer ring part (12). Both the balance box (8) and the attachment box (312) are located on a center line of the pillow body, as shown in FIG. 3 and FIG. 6.

In Embodiment One, a pathway exists between the balance box and the attachment box (312) for placing electric wires.

A specific working principle of the neck pillow provided in this embodiment is as follows:

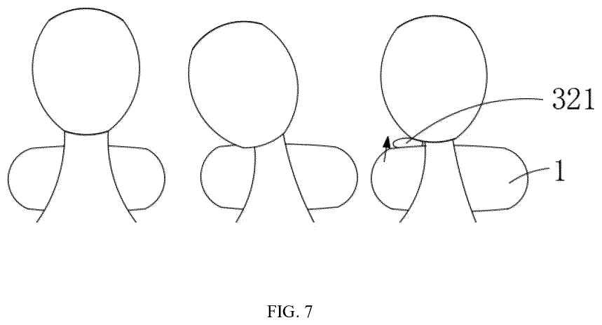

When the user wears the neck pillow, the microphone sensor and the data acquisition collector in the first judgment unit (33) will perform real-time voiceprint recognition to judge whether the user is in the snoring state. When it is detected that the user is snoring, the control system (22) will drive the eccentric vibration motors (311) of the vibration unit (31) to start vibrating. The vibration is transmitted to the user through the attachment box (312) with the arc-shaped surface (3121) that abuts against the rear neck, guiding the user to adjust a sleep posture and alleviating snoring symptoms. At the same time, the pressure sensor or the infrared sensor in the second judgment unit (34) will monitor the head deflection direction of the user in real time. When the head of the user deflects toward the left, the pressure sensor is subjected to pressure or the detection area of the infrared sensor deforms, and the control system (22) will drive the air pump (322) to inflate the first airbag (3211). The first airbag (3211) deforms upward, supporting the neck-cheek junction area of the user, adjusting a support force and a support angle of the neck pillow for the neck of the user, achieving single-side support, and returning the head to a position more conducive to keeping an airway unobstructed, as shown in FIG. 7.

### Embodiment Two

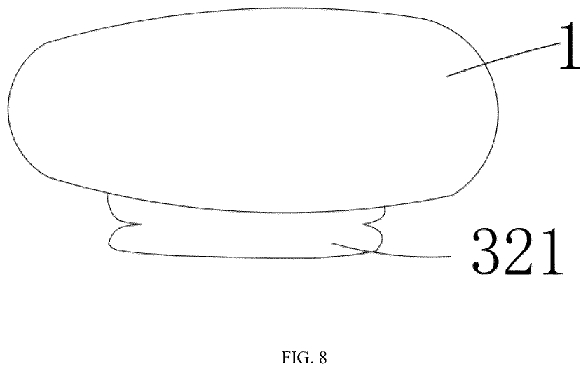

Embodiment Two is basically the same as Embodiment One, but differs in some details. For example: the airbag element (321) is disposed below the pillow main body (1), as shown in FIG. 8.

This embodiment provides a portable neck pillow with an anti-snoring assembly (3), comprising a pillow main body (1) and a control module (2). The anti-snoring assembly (3) comprises a vibration unit (31) and an inflation and deflation unit (32). The inflation and deflation unit (32) comprises an airbag element (321), an air pump (322), and an air path part (323) connecting the airbag element (321) and the air pump (322). And, the anti-snoring assembly (3) further comprises a first judgment unit (33) and a second judgment unit (34). The first judgment unit (33) is used for judging a snoring state of a user. The second judgment unit (34) is used for judging a head deflection direction of the user. The control module (2) comprises a power supply (21) and a control system (22). The control system (22) controls movement of the vibration unit (31) and the inflation and deflation unit (32) according to the first judgment unit (33) and the second judgment unit (34).

In Embodiment Two, the pillow main body (1) is made of a memory foam material.

In Embodiment Two, the portable neck pillow further comprises a massage function. When the massage function is turned on, the control system (22) drives the eccentric vibration motors (311) of the vibration unit (31) to vibrate, and the control system (22) adjusts a motion frequency and an operation duration of the eccentric vibration motors (311).

The foregoing description of several embodiments of the present application has been presented for purposes of illustration. The foregoing description is not intended to be exhaustive, nor is the present application limited to the precise configurations, structures, and/or steps disclosed. Obviously, according to the above teachings, for a person of ordinary skill in the art, several variations and modifications can be made without departing from the concept of the present invention, and these all belong to the protection scope of the present invention. Therefore, the protection scope of the patent of the present invention shall be subject to the appended claims.

What is claimed is:

1. A portable neck pillow with an anti-snoring assembly, comprising a pillow main body and a control module, wherein:
the anti-snoring assembly comprises a vibration unit and an inflation and deflation unit, the inflation and deflation unit comprises an airbag element, an air pump, and an air path part connecting the airbag element and the air pump;
the anti-snoring assembly further comprises a first judgment unit and a second judgment unit, the first judgment unit being for judging a snoring state of a user, and the second judgment unit being for judging a head deflection direction of the user;
the control module comprises a power supply and a control system; and
the control system controls movement of the vibration unit and the inflation and deflation unit according to the first judgment unit and the second judgment unit.

2. The portable neck pillow with an anti-snoring assembly according to claim 1, wherein the airbag element and the pillow main body are in a split-type connection, the pillow main body is sleeved with a first kit, the airbag element is at least partially sleeved with a second kit, and at least a partial connection area between the first kit and the second kit is a detachable connection; and, the airbag element is spatially located above the pillow main body.

3. The portable neck pillow with an anti-snoring assembly according to claim 1, wherein the pillow main body comprises an inner ring part and an outer ring part, a left side and a right side of the outer ring part are respectively provided with a first control compartment and a second control compartment, the air pump is fixed in the first control compartment, the power supply is fixed in the second control compartment, and, the control module further comprises a button, and the button is connected to the second control compartment.

4. The portable neck pillow with an anti-snoring assembly according to claim 1, wherein the vibration unit comprises a plurality of eccentric vibration motors and an attachment box for fixing the eccentric vibration motors, the attachment box comprises at least one arc-shaped surface, and the arc-shaped surface abuts against a rear neck position of a user.

5. The portable neck pillow with an anti-snoring assembly according to claim 1, wherein the airbag element comprises a first airbag and a second airbag, and the first airbag and the second airbag are connected through a connection segment; and, the air path part comprises an air tube, an air nozzle is disposed below the first airbag and the second airbag, and the air nozzle is connected to the air tube.

6. The portable neck pillow with an anti-snoring assembly according to claim 1, wherein the first kit comprises a through hole, and the air tube passes through the through hole and is then connected to the air pump along the outer ring part; and, the inflation and deflation unit further comprises a three-way valve or a two-way valve.

7. The portable neck pillow with an anti-snoring assembly according to claim 1, wherein the first judgment unit comprises a microphone sensor and a data acquisition collector, and the control system controls the first judgment unit to perform real-time voiceprint recognition; and, when the first judgment unit determines that the user is in the snoring state, the control system drives the vibration unit to perform vibration and/or drives the air pump to cause the airbag element to perform inflation and deflation.

8. The portable neck pillow with an anti-snoring assembly according to claim 1, wherein the second judgment unit comprises a pressure sensor, and the pressure sensor is disposed on the inner ring part; and, when a head of a user deflects toward a left or toward a right, the pressure sensor is subjected to pressure, and the control system drives the air pump to cause the airbag element subjected to the pressure to perform inflation.

9. The portable neck pillow with an anti-snoring assembly according to claim 8, wherein the second judgment unit comprises an infrared sensor, and the infrared sensor is disposed on the outer ring part; and, when the head of the user deflects toward the left or toward the right, the pillow main body deforms, a detection area of the infrared sensor deforms, and the control system drives the air pump to cause the airbag element to perform inflation.

10. The portable neck pillow with an anti-snoring assembly according to claim 1, wherein the portable neck pillow further comprises a balance box, the balance box is disposed in a middle area of the outer ring part, and both the balance box and the attachment box are located on a center line of a pillow body.
